(19) United States
(12) Patent Application Publication
(10) Pub. No.: US 2026/0256687 A1
(43) Pub. Date: Sep. 3, 2026

(54) Oral Care Compositions Based On Kocuria And Methods

(71) Applicant: Colgate-Palmolive Company, New York, NY (US)

(72) Inventors: Michael NORTH, Middlesex, NJ (US); Jennifer GRONLUND, Flemington, NJ (US); Lyndsay SCHAEFFER-KORBYLO, Flemington, NJ (US); Carlo DAEP, Avenel, NJ (US)

(73) Assignee: Colgate-Palmolive Company, New York, NY (US)

(21) Appl. No.: 19/164,984

(22) PCT Filed: Mar. 15, 2024
(86) PCT No.: PCT/US2024/020204

§ 371 Date: Sep. 12, 2025

Related U.S. Application Data
(60) Provisional application No. 63/452,287, filed on Mar. 15, 2023.

(51) Int. Cl. A61K 8/99; A61Q 11/00

(52) U.S. Cl. CPC A61K 8/99; A61Q 11/00

(57) ABSTRACT

The disclosure relates to compositions and methods of using bacteria, e.g., bacteria from the genus Kocuria, to impart tooth enamel whitening and/or stain prevention and reduction; and oral care compositions for use in such methods. The disclosure also relates to methods of using oral care compositions, methods of screening, and methods of manufacture.

### FIELD

The field relates to compositions and methods of using bacteria, e.g., bacteria from the genus Kocuria, to impart tooth enamel whitening and/or stain prevention and reduction; and oral care compositions for use in such methods. The field also relates to methods of using oral care compositions, methods of screening, and methods of manufacture.

### BACKGROUND

A tooth is comprised of an inner dentin layer and an outer hard enamel layer that protects the dentin layer. The enamel layer of a tooth is naturally an opaque white or slightly off-white color. The enamel layer can become stained or discolored. The enamel layer of a tooth is composed of hydroxyapatite mineral crystals that create a somewhat porous surface. It is believed that the pores of the enamel layer allow staining agents and discoloring substances to permeate the enamel and discolor the tooth.

Many substances that a person confronts or comes into contact with on a daily basis can “stain” teeth. In particular, foods and fluids such as tea and coffee that one consumes tend to stain one's teeth. These products or substances tend to accumulate on the enamel layer of the tooth and form a pellicle film over the teeth. These staining substances can then permeate the enamel layer. This problem occurs gradually over many years, but imparts a noticeable discoloration of the enamel of one's teeth.

Abrasive agents (for example, silica-based agents) are often used in oral care compositions for stain removal. However, brushing with compositions comprising such agents often leads to enamel damage. This can cause problems such as dentinal hypersensitivity. Peroxide-based agents are also known to have teeth whitening effects. However, peroxide-containing compositions are often unstable and lose whitening efficacy during storage. It is also known to use polyphosphate compounds (for example, pyrophosphates, tripolyphosphates and hexametaphosphates) for stain removal. However, such polyphosphate compounds may interfere with enamel remineralization (i.e. the reformation of crystalline hydroxyapatite—the main constituent of enamel) due to their crystal growth inhibiting properties.

Peroxides are generally deemed safe from a physiological standpoint, and can be effective to whiten teeth. Such peroxides include hydrogen peroxide, carbamide peroxide, sodium perborate, and sodium percarbonate. When these peroxides are in appropriate contact with teeth they will usually oxidize stains, rendering teeth whiter. However, because of stability and the potential interaction with other components, it can be difficult to formulate oral care compositions containing peroxides where the peroxides are compatible with the other ingredients in the formulation. For example, peroxide formulations sometimes need to be in low water compositions to maintain the long-term stability of the peroxide. However, the low-water nature of the composition can affect the palatability and can lead to an adverse consumer experience.

Accordingly, because of the issues that can arise with current oral care compositions that are intended for stain prevention and/or enamel whitening, there is a need for additional oral care formulations.

### SUMMARY OF THE INVENTION

The disclosure contemplates that certain oral care compositions comprising various bacteria can impart teeth whitening. Traditionally whitening is achieved through chemical means to impart abrasive characteristics or bleaching properties. However, the present disclosure indicates that the use of certain bacterial species are able to show a significant improvement in the prophylaxis or reduction of tooth enamel staining. Interestingly, and without being bound by theory, it is believed that this feature may be species dependent and bacteria of the same genus may not always display uniform whitening properties.

In one aspect, the present disclosure provides oral care compositions comprising bacteria from the genus Kocuria that have improved tooth enamel whitening and/or reduction or prophylaxis of enamel staining relative to control formulations.

For example, the disclosure provides an oral care composition (Composition 1.0) comprising an effective amount of at least one bacterial species, e.g., a gram-positive bacterial species, e.g., a bacterial species selected from the genus Kocuria (e.g., Kocuria rhizophila), wherein the amount of the bacterial species is in an amount effective to reduce enamel staining and/or increase enamel whitening in the oral cavity. For example, in various aspects the oral care compositions of the present disclosure include:
1.1 Composition 1.0, wherein the bacterial species is a gram-positive bacterial species.
1.2 Composition 1.0 or 1.1, wherein the gram-positive bacterial species is from the genus Kocuria.
1.3 Composition 1.0, 1.1, or 1.2, wherein the bacterial species is from the genus Kocuria and wherein the bacterial species is selected from the group consisting of: Kocuria aegyptia, Kocuria arsenatis, Kocuria assamensis, Kocuria atrinae, Kocuria carniphila, Kocuria dechangensis, Kocuria flava, Kocuria gwangalliensis, Kocuria halotolerans, Kocuria himachalensis, Kocuria indica, Kocuria koreensis, Kocuria marina, Kocuria oceani, Kocuria ocularis, Kocuria palustris, Kocuria pelophila, Kocuria polaris, Kocuria rhizophila, Kocuria rosea, Kocuria salina, Kocuria salsicia, Kocuria sediminis, Kocuria subflava, Kocuria tuifanensis, Kocuria tytonicola, Kocuria uropygialis, Kocuria uropygioeca, Kocuria varians, and combinations of any of the foregoing species.
1.4 Any of the preceding compositions, wherein the bacterial species is Kocuria rhizophila.
1.5 Any foregoing composition wherein the bacterial species (e.g., a bacterial species selected from the genus Kocuria (e.g., Kocuria rhizophila), is at least 0.1%, e.g., 0.1% to 5%, e.g., about 0.5%, 1% or 2% by weight of the composition.
1.6 Any of the preceding compositions further comprising an anti-calculus agent.
1.7 Any of the preceding compositions further comprising an anti-calculus agent which is a polyphosphate, e.g., pyrophosphate, tripolyphosphate, or hexametaphosphate, e.g., in sodium salt form.
1.8 Any of the preceding compositions comprising at least one surfactant selected from sodium lauryl sulfate, cocamidopropyl betaine, and combinations thereof.
1.9 Any of the preceding compositions comprising an anionic surfactant, e.g., selected from sodium lauryl sulfate, sodium laureth sulfate, and mixtures thereof.
1.10 Any of the preceding compositions comprising sodium lauryl sulfate, in an amount from 0.5-3% by wt of the composition.
1.11 Any of the preceding compositions comprising at least one humectant.
1.12 Any of the preceding compositions comprising at least one humectant selected from glycerin, sorbitol and combinations thereof.
1.13 Any of the preceding compositions comprising at least one polymer.
1.14 Any of the preceding compositions comprising at least one polymer selected from polyethylene glycols, polyvinylmethyl ether maleic acid copolymers, polysaccharides (e.g., cellulose derivatives, for example carboxymethyl cellulose, or polysaccharide gums, for example xanthan gum or carrageenan gum), and combinations thereof.
1.15 Any of the preceding compositions comprising one or more abrasives, e.g., silica, calcium carbonate, or calcium phosphate abrasives.
1.16 Any of the preceding compositions comprising gum strips or fragments.
1.17 Any of the preceding compositions comprising flavoring, fragrance and/or coloring.
1.18 Any composition obtained or obtainable by combining the ingredients as set forth in any of the preceding compositions.
1.19 Any of the preceding oral care compositions, wherein the composition is in the form selected from: a mouthwash, toothpaste, tooth gel, tooth powder, non-abrasive gel, mousse, foam, mouth spray, lozenge, oral tablet, or dental implement.
1.20 Any of the preceding compositions wherein the composition is a toothpaste or a mouthwash.
1.21 Any of the preceding compositions wherein the composition is a toothpaste optionally further comprising one or more of one or more of water, abrasives, surfactants, foaming agents, vitamins, polymers, enzymes, humectants, thickeners, preservatives, flavorings, colorings and/or combinations thereof.
1.22 Any preceding composition, wherein the composition is a toothpaste further comprising water, abrasive, surfactant, humectant, thickener, and flavoring.
1.23 Any preceding composition wherein the composition is a toothpaste obtained or obtainable by a method of mixing with a toothpaste base, e.g., a toothpaste base comprising one or more of one or more of water, abrasives, surfactants, foaming agents, vitamins, polymers, enzymes, humectants, thickeners, antimicrobial agents, preservatives, flavorings, colorings and/or combinations thereof.
1.24 Any preceding composition for use in reducing staining and/or increasing enamel whitening.

For example, the invention provides in one aspect, an oral care composition comprising an effective amount of at least one bacterial species, e.g., a gram-positive bacterial species, e.g., a bacterial species selected from the genus Kocuria (e.g., Kocuria rhizophila), e.g., any of Composition 1.0, et seq. for use in the prophylaxis or reduction of enamel staining and/or increasing enamel whitening.

In another aspect, the disclosure provides a method (Method 1.0) for prophylaxis or reduction of enamel staining and/or increasing enamel whitening, comprising contacting the oral cavity with a composition comprising contacting the oral cavity of a subject in need thereof with an effective amount of at least one bacterial species, e.g., a bacterial species selected from the genus Kocuria (e.g., Kocuria rhizophila), e.g., any of Composition 1.0, et seq, e.g., by brushing, e.g. on a regular basis over a sufficient period of time. For example, in various aspects, Method 1.0 included the following:
1.1 Method 1.0, wherein the bacterial species is a gram-positive bacterial species.
1.2 Method 1.0 or 1.1, wherein the gram-positive bacterial species is from the genus Kocuria.
1.3 Method 1.0, 1.1, or 1.2, wherein the bacterial species is from the genus Kocuria and wherein the species is selected from the group consisting of: Kocuria aegyptia, Kocuria arsenatis, Kocuria assamensis, Kocuria atrinae, Kocuria carniphila, Kocuria dechangensis, Kocuria flava, Kocuria gwangalliensis, Kocuria halotolerans, Kocuria himachalensis, Kocuria indica, Kocuria koreensis, Kocuria marina, Kocuria oceani, Kocuria ocularis, Kocuria palustris, Kocuria pelophila, Kocuria polaris, Kocuria rhizophila, Kocuria rosea, Kocuria salina, Kocuria salsicia, Kocuria sediminis, Kocuria subflava, Kocuria turfanensis, Kocuria tytonicola, Kocuria uropygialis, Kocuria uropygioeca, Kocuria varians, and combinations of any of the foregoing species.
1.4 Any of the preceding methods, wherein the bacterial species is Kocuria rhizophila.
1.5 Any foregoing methods wherein the amount of the bacterial species, e.g., a bacterial species selected from the genus Kocuria (e.g., Kocuria rhizophila), e.g., any of Composition 1.0, et seq, e.g., is at least 0.1%, e.g., 0.1% to 5%, e.g., about 0.5%, 1%, 2% or 5% by weight of the composition.
1.6 Any of the preceding methods further comprising an anti-calculus agent.
1.7 Any of the preceding methods further comprising an anti-calculus agent which is a polyphosphate, e.g., pyrophosphate, tripolyphosphate, or hexametaphosphate, e.g., in sodium salt form.
1.8 Any of the preceding methods comprising at least one surfactant selected from sodium lauryl sulfate, cocamidopropyl betaine, and combinations thereof.
1.9 Any of the preceding methods comprising an anionic surfactant, e.g., selected from sodium lauryl sulfate, sodium laureth sulfate, and mixtures thereof.
1.10 Any of the preceding methods comprising sodium lauryl sulfate, in an amount from 0.5-3% by wt of the composition.
1.11 Any of the preceding methods comprising at least one humectant.
1.12 Any of the preceding methods comprising at least one humectant selected from glycerin, sorbitol and combinations thereof.
1.13 Any of the preceding methods comprising at least one polymer.
1.14 Any of the preceding methods comprising at least one polymer selected from polyethylene glycols, polyvinylmethyl ether maleic acid copolymers, polysaccharides (e.g., cellulose derivatives, for example carboxymethyl cellulose, or polysaccharide gums, for example xanthan gum or carrageenan gum), and combinations thereof.
1.15 Any of the preceding methods comprising one or more abrasives, e.g., silica, calcium carbonate, or calcium phosphate abrasives.
1.16 Any of the preceding methods comprising gum strips or fragments.
1.17 Any of the preceding methods comprising flavoring, fragrance and/or coloring.
1.18 Any of the preceding methods, wherein the composition is a mouthwash, toothpaste, tooth gel, tooth powder, non-abrasive gel, mousse, foam, mouth spray, lozenge, oral tablet, or dental implement.
1.19 Any of the preceding methods wherein the composition is a toothpaste or a mouthwash.
1.20 Any of the preceding methods wherein the composition is a toothpaste optionally further comprising one or more of one or more of water, abrasives, surfactants, foaming agents, vitamins, polymers, enzymes, humectants, thickeners, preservatives, flavorings, colorings and/or combinations thereof.
1.21 Any preceding method, wherein the composition is a toothpaste further comprising water, abrasive, surfactant, humectant, thickener, and flavoring.
1.22 Any preceding method wherein the composition is a toothpaste obtained or obtainable by a method of mixing with a toothpaste base, e.g., a toothpaste base comprising one or more of one or more of water, abrasives, surfactants, foaming agents, vitamins, polymers, enzymes, humectants, thickeners, antimicrobial agents, preservatives, flavorings, colorings and/or combinations thereof.
1.23 Any preceding method for use in reducing staining and/or increasing enamel whitening.

Further provided is a method of preventing or mitigating or treating one or more of tooth decay, caries and/or gum disease, gingivitis, periodontitis, peri-implantitis, peri-implant mucositis, necrotizing gingivitis, necrotizing periodontitis, systemic health disorders and caries in a subject in need thereof, the method comprising contacting an oral cavity of the subject with an oral care composition comprising an effective amount of at least one bacterial species, e.g., a bacterial species selected from the genus Kocuria (e.g., Kocuria rhizophila), e.g., any of Compositions 1, et seq.

In a further aspect, the disclosure provides a use of a bacterial species e.g., a bacterial species selected from the genus Kocuria (e.g., Kocuria rhizophila), e.g., any of Composition 1.0, et seq, in the manufacture of an oral care composition, e.g., any of Composition 1.0, et seq., for use in reducing staining and/or increasing enamel whitening.

In yet another aspect, the methods described herein comprise contacting a dental surface with a bacterial species selected from the genus Kocuria (e.g., Kocuria rhizophila) and an orally acceptable carrier, as defined above. The methods are applicable to dental surfaces of non-human mammals such as companion animals (for example, dogs and cats), as well as to humans. In one embodiment, the dental surface is a surface of a natural tooth of a mammal, for example a human.

In still yet another aspect, the dental surface is substantially free of chemical stains and the present methods are effective to inhibit or prevent the formation and development of new chemical stains, as can occur for example by oral use of tobacco products (including smoking) or by drinking tea or coffee, subsequent to contact with any of Composition 1.0 et seq, or treatment according to any of Method 1.0 et seq. Where the dental surface already possesses some degree of chemical staining, the any of Method 1.0 et seq are effective to inhibit or prevent or reduce further development of the existing stain.

Accordingly, in one embodiment, any of Method 1.0 et seq comprises, after contacting the dental surface with bacterial species selected from the genus Kocuria (e.g., Kocuria rhizophila), exposing the dental surface to a chemical stain-inducing material such as a tobacco product, tea or coffee. Chemical staining resulting from such exposure is, in this embodiment, reduced or inhibited or prevented by the prior contacting of the dental surface with the bacterial species selected from the genus Kocuria (e.g., Kocuria rhizophila). In some embodiments, the dental surface is substantially free of chemical stains prior to exposing to a chemical stain-inducing material as defined above.

In yet another aspect, it is desirable that the bacterial species selected from the genus Kocuria (e.g., Kocuria rhizophila), e.g., any of Composition 1.0 et seq, should remain in contact with the dental surface for a period of time sufficient to provide effective prophylaxis, inhibition, reduction or removal of chemical staining. For example, in one aspect, prophylaxis includes providing a coating that inhibits or reduces the amount of stain from adhering to the dental surface. Depending on various factors including the nature of other materials optionally present in combination with the bacterial species selected from the genus Kocuria (e.g., Kocuria rhizophila), the precise procedure by which contact is effected (e.g., rinsing, brushing, placement of a strip, painting or chewing) and the desired degree and/or duration of inhibition of staining, a suitable minimum period of contact can be from 10 seconds to 8 hours. Where the bacterial species selected from the genus Kocuria (e.g., Kocuria rhizophila) is applied as a component of a mouthwash, an illustrative minimum period of rinsing is from 10 seconds to 2 minutes. Where the bacterial species selected from the genus Kocuria (e.g., Kocuria rhizophila) is applied as a component of a dentifrice, an illustrative minimum period of brushing is from 30 seconds to 5 minutes, or at least 1 minute, or at least 2 minutes. Where the bacterial species selected from the genus Kocuria (e.g., Kocuria rhizophila), e.g., any of Composition 1.0 et seq, is applied as a component of an oral strip, the strip is placed on the dental surface illustratively for a period of from 15 minutes to 8 hours (for example, overnight). Where the bacterial species selected from the genus Kocuria (e.g., Kocuria rhizophila), e.g., any of Composition 1.0 et seq, is applied as a component of a liquid whitener composition, the composition is painted onto the dental surface and left in place illustratively for a period of from 5 minutes to 8 hours (for example, overnight). Where the bacterial species selected from the genus Kocuria (e.g., Kocuria rhizophila), e.g., any of Composition 1.0 et seq, is applied as a component of a chewing gum, an illustrative minimum period of chewing is from 1 to 20 minutes.

Increasing the degree of agitation in the mouth during rinsing, brushing or chewing can lead to improved contact of the bacterial species selected from the genus Kocuria (e.g., Kocuria rhizophila), e.g., any of Composition 1.0 et seq, with the dental surface and enhance the degree of stain prophylaxis, reduction or removal. Thus, in an embodiment where the bacterial species selected from the genus Kocuria (e.g., Kocuria rhizophila) is present as an ingredient of a dentifrice, e.g., any of Composition 1.0 et seq, vigorous brushing with the dentifrice can be particularly effective.

In some embodiments, the bacterial species selected from the genus Kocuria (e.g., Kocuria rhizophila), e.g., any of Composition 1.0 et seq, is applied to the dental surface by means of a dental tray. Accordingly, further provided is a dental tray comprising an oral care composition as defined herein. The dental tray may be formed by procedures that are well-known in the art of oral care. Dental trays are generally formed to correspond to the structure of the dental surface.

In another aspect, the invention relates to methods of screening for compounds that reduce staining and/or increasing enamel whitening, wherein screening steps include:
Determining the ability of a first compound (e.g., test compound) to reduce staining and/or increase enamel whitening, e.g., comparing stain reduction and/or enamel whitening of at least one species of bacteria, e.g., a bacterial species selected from the genus Kocuria (e.g., Kocuria rhizophila), e.g., any of Compositions 1, et seq;
Optionally determining the ability of a second compound (e.g., control compound) to reduce staining and/or whiten enamel;
Optionally comparing the profile of the first compound with the profile of the second compound;
Selecting a test compound for further testing based upon its ability to reduce staining in the oral cavity and/or whiten the enamel, e.g., as compared to the control compound.
   For example, the control compound in the foregoing method of screening may be a gram-positive bacteria, e.g., Kocuria aegyptia, Kocuria arsenatis, Kocuria assamensis, Kocuria atrinae, Kocuria carniphila, Kocuria dechangensis, Kocuria flava, Kocuria gwangalliensis, Kocuria halotolerans, Kocuria himachalensis, Kocuria indica, Kocuria koreensis, Kocuria marina, Kocuria oceani, Kocuria ocularis, Kocuria palustris, Kocuria pelophila, Kocuria polaris, Kocuria rhizophila, Kocuria rosea, Kocuria salina, Kocuria salsicia, Kocuria sediminis, Kocuria subflava, Kocuria turfanensis, Kocuria tytonicola, Kocuria uropygialis, Kocuria uropygioeca, and Kocuria varian. The disclosure further provides the use of a compound identified in such a screening method in any of the foregoing methods and uses.

### DETAILED DESCRIPTION

Unless otherwise indicated, the terms “%” or “percent” when used in connection with an ingredient of the toothpaste compositions of the invention is intended to refer to the percent by weight relative to the total oral care composition.

As used herein, “cleaning” generally refers to the removal of contaminants, dirt, impurities, and/or extraneous matter on a target surface. For example, in the context of oral surfaces, where the surface is tooth enamel, the cleaning may remove at least some of a film or stain, such as plaque biofilm, pellicle or tartar.

As used herein, an “oral care composition” refers to a composition for which the intended use includes oral care, oral hygiene, and/or oral appearance, or for which the intended method of use comprises administration to the oral cavity, and refers to compositions that are palatable and safe for topical administration to the oral cavity, and for providing a benefit to the teeth and/or oral cavity. The term “oral care composition” thus specifically excludes compositions which are highly toxic, unpalatable, or otherwise unsuitable for administration to the oral cavity. In some embodiments, an oral care composition is not intentionally swallowed, but is rather retained in the oral cavity for a time sufficient to affect the intended utility. The oral care compositions as disclosed herein may be used in nonhuman mammals such as companion animals (e.g., dogs and cats), as well as by humans. In some embodiments, the oral care compositions as disclosed herein are used by humans. Oral care compositions include, for example, dentifrice and mouthwash. In some embodiments, the disclosure provides mouthwash formulations.

As used herein, “orally acceptable” refers to a material that is safe and palatable at the relevant concentrations for use in an oral care formulation, such as a mouthwash or dentifrice.

As used herein, “orally acceptable carrier” refers to any vehicle useful in formulating the oral care compositions disclosed herein. The orally acceptable carrier is not harmful to a mammal in amounts disclosed herein when retained in the mouth, without swallowing, for a period sufficient to permit effective contact with a dental surface as required herein. In general, the orally acceptable carrier is not harmful even if unintentionally swallowed. Suitable orally acceptable carriers include, for example, one or more of the following: water, a thickener, a buffer, a humectant, a surfactant, an abrasive, a sweetener, a flavorant, a pigment, a dye, an anti-caries agent, an anti-bacterial, a whitening agent, a desensitizing agent, a vitamin, a preservative, an enzyme, and mixtures thereof.

In some aspects, the oral care compositions disclosed herein, e.g., Composition 1.0, et seq., comprise water. Water employed in the preparation of the oral care compositions disclosed herein, e.g., Composition 1.0, et seq., should be deionized and free of organic impurities. Water may make up the balance of the oral care composition. In some embodiments, the oral care compositions disclosed herein, e.g., Composition 1.0, et seq., comprise 0 to 90 weight % water, e.g., 0.1 to 90 weight % water, e.g., 1 to 80 weight % water, e.g., 2 to 70 weight % water, 5 to 60 weight % water, e.g., 5 to 50 weight % water, e.g., 20 to 60 weight % water, e.g., 10 to 40 weight % water. This amount of water includes the free water that is added plus that amount which is introduced with other components of the oral care composition, such as with sorbitol.

A thickener provides a desirable consistency and/or stabilizes and/or enhances performance (e.g., provides desirable active release characteristics upon use) of the oral care composition. In some embodiments, the oral care compositions disclosed herein, e.g., Composition 1.0, et seq., comprise from 0.01 to 15 weight % of a thickener, 0.1 to 15 weight % of a thickener, e.g., 0.1 to 10 weight % of a thickener, e.g., 0.1 to 5 weight % of a thickener, e.g., 0.5 to 10 weight % of a thickener, e.g., 0.5 to 5 weight % of at a thickener, e.g., 1 to 4 weight % of a thickener, e.g., 2 to 5 weight % of a thickener, e.g., 2 to 4 weight % of a thickener, e.g., 3 to 4 weight % of a thickener. Higher weight percentages may be used for chewing gums, lozenges and breath mints, sachets, non-abrasive gels and subgingival gels. Thickeners that may be used in the oral care compositions disclosed herein, e.g., Composition 1.0, et seq., include, for example, carboxyvinyl polymers, carrageenan (also known as carrageenan gum), hydroxyethyl cellulose (HEC), natural and synthetic clays (e.g., Veegum and laponite), water soluble salts of cellulose ethers (e.g., sodium carboxymethylcellulose (CMC) and sodium carboxymethyl hydroxyethyl cellulose), natural gums (e.g., gum karaya, xanthan gum, gum arabic, and gum tragacanth), colloidal magnesium aluminum silicate, silica (e.g., finely divided silica), cross-linked poly(vinyl)pyrrolidone, carbowaxes, fatty acids and salts thereof (e.g., stearic acid and palmitic acid), fatty alcohols (e.g., stearyl alcohol), and mixtures thereof. In some embodiments, a mixture of thickening silica and carrageenan gum is used as the thickener in the oral care compositions disclosed herein, e.g., Composition 1.0, et seq. In some embodiments, the oral care compositions disclosed herein, e.g., Composition 1.0, et seq., comprise from 0.01 to 15 weight % of thickening silica and carrageenan gum, 0.1 to 15 weight % of thickening silica and carrageenan gum, e.g., 0.1 to 10 weight % of thickening silica and carrageenan gum, e.g., 0.1 to 5 weight % of thickening silica and carrageenan gum, e.g., 0.5 to 10 weight % of thickening silica and carrageenan gum, e.g., 0.5 to 5 weight % of thickening silica and carrageenan gum, e.g., 1 to 4 weight % of thickening silica and carrageenan gum, e.g., 2 to 5 weight % of thickening silica and carrageenan gum, e.g., 2 to 4 weight % of thickening silica and carrageenan gum, e.g., 3 to 4 weight % of thickening silica and carrageenan gum.

A buffer adjusts the pH of oral care compositions, for example, to a range of about pH 4.0 to about pH 9.5. In some embodiments, the oral care compositions disclosed herein, e.g., Composition 1.0, et seq., comprise from 0.1 to 10 weight % of a buffer, 0.5 to 10 weight % of a buffer, e.g., 0.5 to 5 weight % of a buffer, e.g., 0.5 to 4 weight % of a buffer, e.g., 0.5 to 3 weight % of a buffer, e.g., 0.5 to 2 weight % of a buffer, e.g., 1 to 2 weight % of a buffer. Buffers that may be used in the oral care compositions disclosed herein, e.g., Composition 1.0, et seq., include, for example, sodium bicarbonate, sodium phosphate {e.g., monosodium phosphate (NaH2PO4), disodium phosphate (Na2HPO4), trisodium phosphate (Na3PO4)}, sodium hydroxide, sodium carbonate, sodium acid pyrophosphate, citric acid, sodium citrate, and mixtures thereof. In some embodiments, sodium hydroxide is used as the buffer in the oral care compositions disclosed herein, e.g., Composition 1.0, et seq. In some embodiments, the oral care compositions disclosed herein, e.g., Composition 1.0, et seq., comprise from 0.1 to 10 weight % of sodium hydroxide, e.g., 0.5 to 10 weight % of sodium hydroxide, e.g., 0.5 to 5 weight % of sodium hydroxide, e.g., 0.5 to 4 weight % of sodium hydroxide, e.g., 0.5 to 3 weight % of sodium hydroxide, e.g., 0.5 to 2 weight % of sodium hydroxide, e.g., 1 to 2 weight % of sodium hydroxide.

A humectant keeps oral care compositions from hardening upon exposure to air. Certain humectants can also impart desirable sweetness or flavor to oral care compositions. In some embodiments, the oral care compositions disclosed herein, e.g., Composition 1.0, et seq., comprise, on a pure humectant basis, from 0 to 70 weight % of a humectant, e.g., 10 to 70 weight % of a humectant, e.g., 10 to 65 weight % of a humectant, e.g., 10 to 60 weight % of a humectant, e.g., 10 to 50 weight % of a humectant, e.g., 20 to 50 weight % of at a humectant, e.g., 20 to 40 weight % of a humectant. Humectants that may be used in the oral care compositions disclosed herein, e.g., Composition 1.0, et seq., include, for example, glycerin, sorbitol, xylitol, butylene glycol, polyethylene glycol, propylene glycol, trimethyl glycine, and mixtures thereof. In some embodiments, a mixture of glycerin, sorbitol, and propylene glycol is used as the humectant in the oral care compositions disclosed herein, e.g., Composition 1.0, et seq. In some embodiments, the oral care compositions disclosed herein, e.g., Composition 1.0, et seq., comprise, on a pure humectant basis, from 0 to 70 weight % of glycerin, sorbitol, and propylene glycol, e.g., 10 to 70 weight % of glycerin, sorbitol, and propylene glycol, e.g., 10 to 65 weight % of glycerin, sorbitol, and propylene glycol, e.g., 10 to 60 weight % of glycerin, sorbitol, and propylene glycol, e.g., 10 to 50 weight % of glycerin, sorbitol, and propylene glycol, e.g., 20 to 50 weight % of glycerin, sorbitol, and propylene glycol, e.g., 20 to 40 weight % of glycerin, sorbitol, and propylene glycol.

In some embodiments, the oral care compositions disclosed herein, e.g., Composition 1.0, et seq., comprise a surfactant, e.g., selected from anionic, cationic, zwitterionic, and nonionic surfactants, and mixtures thereof. In some embodiments, the surfactant is reasonably stable throughout a wide pH range. In some embodiments, the oral care compositions disclosed herein, e.g., Composition 1.0, et seq., comprise from 0.01 to 10 weight % of a surfactant, e.g., 0.05 to 5 weight % of a surfactant, e.g., 0.1 to 10 weight % of a surfactant, e.g., 0.1 to 5 weight % of a surfactant, e.g., 0.1 to 2 weight % of a surfactant, e.g., 0.5 to 2 weight % of a surfactant. In some embodiments, the oral care compositions disclosed herein, e.g., Composition 1.0, et seq., comprise from 0.01 to 10 weight % of an anionic surfactant, e.g., 0.05 to 5 weight % of an anionic surfactant, e.g., 0.1 to 10 weight % of an anionic surfactant, e.g., 0.1 to 5 weight % of an anionic surfactant, e.g., 0.1 to 2 weight % of an anionic surfactant, e.g., 0.5 to 2 weight % of an anionic surfactant, e.g., 1.5 weight % of an anionic surfactant.

Anionic surfactants that may be used in the oral care compositions disclosed herein, e.g., Composition 1.0, et seq., include, for example,
i. water-soluble salts of higher fatty acid monoglyceride monosulfates, such as the sodium salt of the monosulfated monoglyceride of hydrogenated coconut oil fatty acids such as sodium N-methyl N-cocoyl taurate, sodium cocomonoglyceride sulfate,
ii. higher alkyl sulfates, such as sodium lauryl sulfate,
iii. higher alkyl-ether sulfates, e.g., of formula CH₃(CH₂)ₘCH₂(OCH₂CH₂)ₙOSO₃X, wherein m is 6-16, e.g., 10, n is 1-6, e.g., 2, 3 or 4, and X is Na or K, for example sodium laureth-2 sulfate, CH₃(CH₂)₁₀CH₂(OCH₂CH₂)₂OSO₃Na,
iv. higher alkyl aryl sulfonates such as sodium dodecyl benzene sulfonate (sodium lauryl benzene sulfonate), and
v. higher alkyl sulfoacetates, such as sodium lauryl sulfoacetate (dodecyl sodium sulfoacetate), higher fatty acid esters of 1,2 dihydroxy propane sulfonate, sulfocolaurate (N-2-ethyl laurate potassium sulfoacetamide) and sodium lauryl sarcosinate.

As used herein, “higher alkyl” refers to C6-30 alkyl.

In some embodiments, the oral care compositions disclosed herein, e.g., Composition 1.0, et seq., comprise an anionic surfactant. In some embodiments, the anionic surfactant is the water soluble salt of alkyl sulfates having from 10 to 18 carbon atoms in the alkyl radical and water soluble salts of sulfonated monoglycerides of fatty acids having from 10 to 18 carbon atoms. Sodium lauryl sulfate, sodium lauroyl sarcosinate, and sodium coconut monoglyceride sulfonates are examples of anionic surfactants of that type. In some embodiments, the oral care compositions disclosed herein, e.g., Composition 1.0, et seq., comprise sodium lauryl sulfate, sodium ether lauryl sulfate, or a mixture thereof. In some embodiments, the oral care compositions disclosed herein, e.g., Composition 1.0, et seq., comprise sodium lauryl sulfate. In some embodiments, the oral care compositions disclosed herein, e.g., Composition 1.0, et seq., comprise from 0.01 to 10 weight % sodium lauryl sulfate, e.g., 0.05 to 5 weight % sodium lauryl sulfate, e.g., 0.1 to 10 weight % sodium lauryl sulfate, e.g., 0.1 to 5 weight %, e.g., sodium lauryl sulfate, e.g., 0.1 to 2 weight % sodium lauryl sulfate, e.g., 0.5 to 2 weight % sodium lauryl sulfate, e.g., 1.5 weight % sodium lauryl sulfate.

An abrasive removes debris and surface stains. In some embodiments, the oral care compositions disclosed herein, e.g., Composition 1.0, et seq., comprise 5 to 70 weight % of an abrasive, e.g., 5 to 60 weight % of an abrasive, e.g., 5 to 50 weight % of an abrasive, e.g., 5 to 40 weight % of an abrasive, e.g., 5 to 30 weight % of an abrasive, e.g., 10 to 30 weight % of an abrasive, e.g., 10 to 20 weight % of an abrasive.

Abrasives that may be used in the oral care compositions disclosed herein, e.g., Composition 1.0, et seq., include, for example, a calcium phosphate abrasive, e.g., tricalcium phosphate (Ca₃(PO₄)2), hydroxyapatite (Ca₁₀(PO₄)6(OH)2), dicalcium phosphate dihydrate (CaHPO₄·2H₂O, also sometimes referred to herein as DiCal), calcium pyrophosphate, and mixtures thereof. Calcium carbonate, e.g., precipitated calcium carbonate, may also be employed as an abrasive.

Other abrasives that may be used in the oral care compositions disclosed herein, e.g., Composition 1.0, et seq., include, for example, silica abrasives such as precipitated silicas having a mean particle size of up to about 20 microns, such as Zeodent 115@, marketed by J. M. Huber, as well as sodium metaphosphate, potassium metaphosphate, aluminum silicate, calcined alumina, bentonite or other siliceous materials, or mixtures thereof. Silica abrasives used herein, as well as the other abrasives, may have an average particle size ranging between about 0.1 and about 30 microns, e.g., between about 5 and about 15 microns. The silica abrasives may be from precipitated silica or silica gels, such as silica xerogels. Particular silica xerogels are marketed under the trade name Syloid® by the W. R. Grace & Co. Davison Chemical Division. Precipitated silica materials include those marketed by the J. M. Huber Corp. under the trade name Zeodent®, including the silica carrying the designation Zeodent 115 and 119.

In some embodiments, abrasives that may be used in the oral care compositions disclosed herein, e.g., Composition 1.0, et seq., include silica gels and precipitated amorphous silica having an oil absorption value of about less than about 100 cc/100 g silica and in the range of about 45 cc/100 g to about 70 cc/100 g silica. Oil absorption values are measured using the ASTA Rub-Out Method D281. In some embodiments, the silica comprises colloidal particles having an average particle size of about 3 microns to about 12 microns, and about 5 to about 10 microns.

In some embodiments, the abrasive comprises a large fraction of very small particles, e.g., having a d50 less than about 5 microns, e.g., small particle silica (SPS) having a d50 of about 3 to about 4 microns, e.g., Sorbosil AC AC43® (Ineos). Such small particles may be used in formulations targeted at reducing hypersensitivity. The small particle component may be present in combination with a second larger particle abrasive.

Low oil absorption silica abrasives that may be used in the oral care compositions disclosed herein, e.g., Composition 1.0, et seq., are marketed under the trade designation Sylodent WXA® by Davison Chemical Division of W.R. Grace & Co., Baltimore, Md. 21203. Sylodent 650 XWA®, a silica hydrogel composed of particles of colloidal silica having a water content of about 29% by weight averaging about 7 to about 10 microns in diameter, and an oil absorption of less than about 70 cc/100 g of silica is an example of a low oil absorption silica abrasive that may be used in the oral care compositions disclosed herein, e.g., Composition 1.0, et seq.

In some embodiments, the oral care composition disclosed herein, e.g., Composition 1, e.g, 1.1-1.40, comprise a high cleaning silica. In some embodiments, the oral care compositions disclosed herein, e.g., Composition 1.0, et seq., comprise 5 to 70 weight % high cleaning silica, e.g., 5 to 60 weight % high cleaning silica, e.g., 5 to 50 weight % high cleaning silica, e.g., 5 to 40 weight % high cleaning silica, e.g., 5 to 30 weight % high cleaning silica, e.g., 10 to 30 weight % high cleaning silica, e.g., 10 to 20 weight % high cleaning silica.

In some embodiments, the oral care compositions disclosed herein, e.g., Composition 1.0, et seq., comprise a sweetener. In some embodiments, the oral care compositions disclosed herein, e.g., Composition 1.0, et seq., comprise 0.005 to 10 weight % of a sweetener, e.g., 0.01 to 10 weight % of a sweetener, e.g., 0.1 to 10 weight % of a sweetener, e.g., from 0.1 to 5 weight % of a sweetener, e.g., from 0.1 to 3 weight % of a sweetener, e.g., from 0.1 to 1 weight % of a sweetener, e.g., from 0.1 to 0.5 weight % of a sweetener. Sweeteners that may be used in the oral care compositions disclosed herein, e.g., Composition 1.0, et seq., include, for example, sucrose, glucose, saccharin, sucralose, dextrose, levulose, lactose, mannitol, sorbitol, fructose, maltose, xylitol, saccharin salts (e.g., sodium saccharin), thaumatin, aspartame, D-tryptophan, dihydrochalcones, acesulfame, cyclamate salts, and mixtures thereof. In some embodiments, sodium saccharin is used as the sweetener in the oral care compositions disclosed herein, e.g., Composition 1.0, et seq. In some embodiments, the oral care compositions disclosed herein, e.g., Composition 1.0, et seq., comprise 0.005 to 10 weight % sodium saccharin, e.g., 0.01 to 10 weight % sodium saccharin, e.g., 0.1 to 10 weight % sodium saccharin, e.g., from 0.1 to 5 weight % sodium saccharin, e.g., from 0.1 to 3 weight % sodium saccharin, e.g., from 0.1 to 1 weight % sodium saccharin, e.g., from 0.1 to 0.5 weight % sodium saccharin.

In some embodiments, the oral care compositions disclosed herein, e.g., Composition 1.0, et seq., comprise a flavorant. In some embodiments, the oral care compositions disclosed herein, e.g., Composition 1.0, et seq., comprise 0.1 to 5 weight % of a flavorant, e.g., 0.1 to 4 weight % of a flavorant, e.g., 0.1 to 3 weight % of a flavorant, e.g., 0.1 to 2 weight % of a flavorant, e.g., 0.5 to 2 weight % of a flavorant, e.g., 0.6 to 2 weight % of a flavorant, e.g., 0.7 to 2 weight % of a flavorant, e.g., 0.8 to 2 weight % of a flavorant e.g., 0.9 to 2 weight % of a flavorant, e.g., 1 to 2 weight % of a flavorant. Flavorants that may be used in the oral care compositions disclosed herein, e.g., Composition 1.0, et seq., include, for example, essential oils, as well as various flavoring aldehydes, esters, alcohols, and similar materials, as well as menthol, carvone, and anethole, as well as mixtures thereof. Examples of essential oils include oils of spearmint, peppermint, wintergreen, sassafras, clove, sage, eucalyptus, marjoram, cinnamon, lemon, lime, grapefruit, and orange. In some embodiments, a mixture of peppermint oil and spearmint oil is used as the flavorant in the oral care compositions disclosed herein, e.g., Composition 1.0, et seq.

In some embodiments, the oral care compositions disclosed herein, e.g., Composition 1.0, et seq., comprise a pigment. In some embodiments, the oral care compositions disclosed herein, e.g., Composition 1.0, et seq., comprise 0.001 to 20 weight % of a pigment, e.g., 0.01 to 20 weight % of a pigment, e.g., 0.01 to 20 weight % of a pigment, e.g., 0.1 to 20 weight % of a pigment, e.g., 0.1 to 10 weight % of a pigment, e.g., 0.1 to 5 weight % of a pigment, e.g., 0.1 to 3 weight % of a pigment, e.g., 0.1 to 1 weight % of a pigment. In some embodiments, the oral care compositions disclosed herein, e.g., Composition 1.0, et seq., comprise titanium dioxide. In some embodiments, the oral care compositions disclosed herein, e.g., Composition 1.0, et seq., comprise 0.001 to 20 weight % titanium dioxide, e.g., 0.01 to 20 weight % titanium dioxide, e.g., 0.01 to 20 weight % titanium dioxide, e.g., 0.1 to 20 weight % titanium dioxide, e.g., 0.1 to 10 weight % titanium dioxide, e.g., 0.1 to 5 weight % titanium dioxide, e.g., 0.1 to 3 weight % titanium dioxide, e.g., 0.1 to 1 weight % titanium dioxide.

In some embodiments, the oral care compositions disclosed herein, e.g., Composition 1.0, et seq., further comprise an anti-caries agent. In some embodiments, the oral care compositions disclosed herein, e.g., Composition 1.0, et seq., comprise 0.005 to 10 weight % of the anti-caries agent, e.g., 0.01 to 10 weight % of the anti-caries agent, e.g., 0.01 to 5 weight % of the anti-caries agent, e.g., 0.01 to 1 weight % of the anti-caries agent, e.g., 0.01 to 0.3 weight % of the anti-caries agent, e.g., 0.1 to 10 weight % of the anti-caries agent, e.g., 0.1 to 5 weight % of the anti-caries agent, e.g., 0.1 to 2 weight % of the anti-caries agent, e.g., 0.1 to 1 weight % of the anti-caries agent, e.g., 0.1 to 0.8 weight % of the anti-caries agent, e.g., 0.1 to 0.6 weight % of the anti-caries agent, e.g., 0.1 to 0.5 weight % of the anti-caries agent. In some embodiments, the anti-caries agent is a fluoride ion source. In some embodiments, the oral care compositions disclosed herein, e.g., Composition 1.0, et seq., further comprise 0.005 to 10 weight % of the anti-caries agent which is a fluoride ion source, e.g., 0.01 to 10 weight % of the anti-caries agent which is a fluoride ion source, e.g., 0.01 to 5 weight % of the anti-caries agent which is a fluoride ion source, e.g., 0.01 to 1 weight % of the anti-caries agent which is a fluoride ion source, e.g., 0.01 to 0.3 weight % of the anti-caries agent which is a fluoride ion source, e.g., 0.1 to 10 weight % of the anti-caries agent which is a fluoride ion source, e.g., 0.1 to 5 weight % of the anti-caries agent which is a fluoride ion source, e.g., 0.1 to 2 weight % of the anti-caries agent which is a fluoride ion source, e.g., 0.1 to 1 weight % of the anti-caries agent which is a fluoride ion source, e.g., 0.1 to 0.8 weight % of the anti-caries agent which is a fluoride ion source, e.g., 0.1 to 0.6 weight % of the anti-caries agent which is a fluoride ion source, e.g., 0.1 to 0.5 weight % of the anti-caries agent which is a fluoride ion source. Examples of fluoride ion sources that may be used in the oral compositions disclosed herein, e.g., Composition 1.0, et seq., are found in U.S. Pat. No. 3,535,421 to Briner et al.; U.S. Pat. No. 4,885,155 to Parran, Jr. et al., and U.S. Pat. No. 3,678,154 to Widder et al, incorporated herein by reference in their entirety. Other examples of fluoride ion sources include, for example, stannous fluoride, sodium fluoride, potassium fluoride, sodium monofluorophosphate, sodium fluorosilicate, ammonium fluorosilicate, amine fluoride (e.g., N′-octadecyltrimethylendiamine-N,N,N′-tris(2-ethanol)-dihydrofluoride), ammonium fluoride, titanium fluoride, hexafluorosulfate, and combinations thereof. In certain embodiments the fluoride ion source includes stannous fluoride, sodium fluoride, and sodium monofluorophosphate, as well as mixtures thereof. In some embodiments, the anti-caries agent is sodium fluoride. In some embodiments, the oral care compositions disclosed herein, e.g., Composition 1.0, et seq., comprise 0.005 to 10 weight % sodium fluoride, e.g., 0.01 to 10 weight % sodium fluoride, e.g., 0.01 to 5 weight % sodium fluoride, e.g., 0.01 to 1 weight % sodium fluoride, e.g., 0.01 to 0.3 weight % sodium fluoride, e.g., 0.1 to 10 weight % sodium fluoride, e.g., 0.1 to 5 weight % sodium fluoride, e.g., 0.1 to 2 weight % sodium fluoride, e.g., 0.1 to 1 weight % sodium fluoride, e.g., 0.1 to 0.8 weight % sodium fluoride, e.g., 0.1 to 0.6 weight % sodium fluoride, e.g., 0.1 to 0.5 weight % sodium fluoride.

In some embodiments, the oral care compositions disclosed herein, e.g., Composition 1.0, et seq., comprise the anti-caries agent which is a fluoride ion source in an amount sufficient to supply 25 ppm to 25,000 ppm of fluoride ions, e.g., from 100 to 20,000 ppm of fluoride ions, e.g., from 300 to 15,000 ppm of fluoride ions, e.g., from 500 to 10,000 ppm of fluoride ions, e.g., from 500 to 8,000 ppm of fluoride ions, e.g., from 500 to 6,000 ppm of fluoride ions, e.g., from 500 to 4,000 ppm of fluoride ions, e.g., from 500 to 2,000 ppm of fluoride ions, e.g., from 500 to 1,800 ppm of fluoride ions, e.g., from 1000 to 1600 ppm, e.g., 1450 ppm of fluoride ions. The appropriate level of fluoride ions will depend on the particular application. In some embodiments, toothpaste for consumer use comprises the anti-caries agent which is a fluoride ion source in an amount sufficient to supply from 1,000 to 1,500 ppm of fluoride ions, with pediatric toothpaste having somewhat less. In some embodiments, a dentifrice or coating for professional application comprises the anti-caries agent which is a fluoride ion source in an amount sufficient to supply from 5,000 to 25,000 ppm of fluoride ions.

A whitening agent whitens a tooth to which it is applied. In some embodiments, the oral care compositions disclosed herein, e.g., Composition 1.0, et seq., comprise a whitening agent. In some embodiments, the oral care compositions disclosed herein, e.g., Composition 1.0, et seq., comprise a whitening agent in a dental surface-whitening effective amount, e.g., 0.1 to 90 weight % whitening agent, e.g., 0.5 to 50 weight % whitening agent, e.g., 1 to 30 weight % whitening agent, e.g., 2 to 10 weight % whitening agent. Examples of whitening agents that may be used in the oral compositions disclosed herein, e.g., Composition 1.0, et seq., include, for example, peroxides, metal chlorites, perborates, percarbonates, peroxyacids, hypochlorites, and mixtures thereof. In some embodiments, the whitening agent is hydrogen peroxide or a hydrogen peroxide source, for example, urea peroxide or a peroxide salt or complex (for example, peroxyphosphate, peroxycarbonate, perborate, peroxysilicate, or persulphate salts; for example calcium peroxyphosphate, sodium perborate, sodium carbonate peroxide, sodium peroxyphosphate, and potassium persulfate), or a hydrogen peroxide polymer complex (for example, a peroxide-polyvinyl pyrrolidone polymer complex).

In one aspect the whitening effects of a composition may be quantified using a measurement of the L*a*b* color space. (L*a*b* refers to stain score in accordance with the Commission International de L'Eclairage Laboratory (CIELAB) color scale. L* represents lightness/darkness, a* represents red-green chroma, and b* represents yellow-blue chroma). From measurement of the L*a*b* values, the whiteness index is reported as AWIO, where AWIO can be calculated from L*, a*, b* values, where (x, y) are the chromaticity coordinates of the sample and (xn, yn) is the reference sample:

$$X = {X}_{n} * {( ( L + 16 ) / 116 + a / 500 )}^{3} , {X}_{n} = 95.$$

$$Y = {Y}_{n} * {( ( L + 16 ) / 116 )}^{3} , {Y}_{n} = 100.$$

$$Z = {Z}_{n} * {( ( L + 16 ) / 116 - b / 200 )}^{3} , {Z}_{n} = 108.9$$

$$WIO = Y + 1075.012 ( {x}_{n} - x ) + 145.516 ( {y}_{n} - y )$$

The more positive the value of AWIO* will then indicate an increase in tooth whitening.

L*a*b* values can be measured using an optic shade-taking system to analyze and identify the color of a substrate (or dental surface).

The following examples are further illustrative of the nature of the present invention, but it is understood that the invention is not limited thereto. All amounts and proportions referred to herein and in the appended claims are by weight, unless otherwise indicated.

### EXAMPLES

### Example 1—Efficacy in Prophylaxis or Reduction of Staining

Baseline L, A, B, WIO measurements of 200 human teeth are measured using a spectroshade micro II (Medical High Technologies) and split into 2 treatment groups consisting of 50 teeth per group. After the baseline is measured each individual cell is placed into a 37° C. incubator and continuously cycled in and out of air/stain broth for 1 week.

The stain broth consists of 200 ml of tobacco juice (10% by weight), 200 ml of coffee (lg per 50 ml water), 200 ml of tea (1 bag per 50 ml water), 200 ml of filtered foxhorn cabernet sauvignon wine, 1 L of Trypticase soy broth, 3.75 g gastric mucin, and 0.2 g of FeCl3. The pH of the stain broth is adjusted to between 6.9-7.2 prior to teeth exposure. The broth is replaced every two days. The broth for the two cells differs only in the lack/presence of bacteria. One cell has no bacteria deliberately added and a second has 45 ml of Kocuria rhizophila added to the stain broth prior to staining. After one week the teeth are removed, rinsed, patted dry, and the L, A, B measured. Final WIO measurements and differences are calculated. The results are described in Table 1.

TABLE 1
ΔWIO values for staining efficacy of stain broth vs. human teeth.
Letters (A, B) indicate values statistically different from each other.

| | Average | Standard | |
|---|---|---|---|
| Sample | delta WIO | deviation | 95% confidence |
| Control | −23.5 (A) | 10.9 | 3.0 |
| +Kocuria | −14.8 (B) | 8.0 | 2.2 |
| rhizophilia | | | |

Table 1 also demonstrates that Kocuria rhizophila is capable of providing a stain reduction benefit as the average delta WIO (i.e., change in whitening effects) is statistically less than the control sample where no bacteria is added to the cells (i.e., less stain has accumulated).

1. An oral care composition comprising an effective amount of a bacterial species selected from the genus Kocuria, wherein the amount of the bacterial species is in an amount effective to reduce enamel staining and/or increase enamel whitening in the oral cavity.

2. The oral care composition of claim 1, wherein the bacterial species is from the genus Kocuria and wherein the bacterial species is selected from one or more of the group consisting of: Kocuria aegyptia, Kocuria arsenatis, Kocuria assamensis, Kocuria atrinae, Kocuria carniphila, Kocuria dechangensis, Kocuria flava, Kocuria gwangalliensis, Kocuria halotolerans, Kocuria himachalensis, Kocuria indica, Kocuria koreensis, Kocuria marina, Kocuria oceani, Kocuria ocularis, Kocuria palustris, Kocuria pelophila, Kocuria polaris, Kocuria rhizophila, Kocuria rosea, Kocuria salina, Kocuria salsicia, Kocuria sediminis, Kocuria subflava, Kocuria turfanensis, Kocuria tytonicola, Kocuria uropygialis, Kocuria uropygioeca, Kocuria varians, and combinations of any of the foregoing species.

3. The oral care composition of claim 2, wherein the bacterial species is Kocuria rhizophila.

4. The oral care composition of claim 1, wherein the bacterial species is present in an amount from 0.1% to 5%, by weight of the composition.

5. The oral care composition of claim 1, further comprising an anti-calculus agent.

6. The oral care composition of claim 1, further comprising an anti-calculus agent which is a polyphosphate.

7. The oral care composition of claim 1, comprising at least one surfactant selected from sodium lauryl sulfate, cocamidopropyl betaine, and combinations thereof.

8. The oral care composition of claim 1, wherein the composition comprises gum strips or fragments.

9. The oral care composition of claim 1, wherein the composition is in a form selected from a mouthwash, toothpaste, tooth gel, tooth powder, non-abrasive gel, mousse, foam, mouth spray, lozenge, oral tablet, and dental implement.

10. A method for prophylaxis or reduction of enamel staining and/or increasing enamel whitening in the oral cavity, wherein the method comprises contacting the oral cavity of a subject in need thereof with a composition comprising an effective amount of the composition of claim 1.

11. The method of claim 10, wherein the bacterial species is from the genus Kocuria and wherein the species is selected from the group consisting of: Kocuria aegyptia, Kocuria arsenatis, Kocuria assamensis, Kocuria atrinae, Kocuria carniphila, Kocuria dechangensis, Kocuria flava, Kocuria gwangalliensis, Kocuria halotolerans, Kocuria himachalensis, Kocuria indica, Kocuria koreensis, Kocuria marina, Kocuria oceani, Kocuria ocularis, Kocuria palustris, Kocuria pelophila, Kocuria polaris, Kocuria rhizophila, Kocuria rosea, Kocuria salina, Kocuria salsicia, Kocuria sediminis, Kocuria subflava, Kocuria turfanensis, Kocuria tytonicola, Kocuria uropygialis, Kocuria uropygioeca, Kocuria varians, and combinations of any of the foregoing species.

12. The method of claim 11, wherein the bacterial species is Kocuria rhizophila.

13. The method of claim 11, wherein the Kocuria rhizophila is present from 0.1% to 5% by weight of the composition.

14. The method of claim 11, further comprising an anti-calculus agent.

15. The method of claim 11, comprising at least one surfactant selected from sodium lauryl sulfate, cocamidopropyl betaine, and combinations thereof.

16. The method of claim 11, further comprising at least one humectant.

17. The method of claim 11, further comprising at least one humectant selected from glycerin, sorbitol and combinations thereof.

18. The method of claim 11, wherein the composition is in a form selected from: a mouthwash, toothpaste, tooth gel, tooth powder, non-abrasive gel, mousse, foam, mouth spray, lozenge, oral tablet, and dental implement.
